(19) United States
(12) Patent Application Publication
(10) Pub. No.: US 2019/0374394 A1
(43) Pub. Date: Dec. 12, 2019

(54) Ear/Nose Cleaning Device

(71) Applicant: Cherryl Mbuh Diom, Frisco, TX (US)

(72) Inventor: Cherryl Mbuh Diom, Frisco, TX (US)

(21) Appl. No.: 16/001,938

(22) Filed: Jun. 7, 2018

(51) Int. Cl. A61F 13/38; A61B 17/24; A61B 17/30

(52) U.S. Cl. CPC A61F 13/38; A61F 11/006; A61B 17/30; A61B 17/24

(57) ABSTRACT

An ear/nose cleaning device comprises of a plastic framework, having a pair of arms/handles, a pair of scoops/bulbous-shaped portion at the distal end of the arms/handles, and a U-shaped swab at proximal end of device. Cotton (or other absorbent material) is tightly and finely adhered to plastic body/framework of scoop and U-shaped swab. The scoops have a hollow inside and flat oval edges for scraping and collecting ear wax/debris or nasal mucous and depositing within the hollow portion. Each arm has a gripping/roughen portion to the lateral side to prevent slipping of device when in use.

This application claims the benefit of U.S. Provisional Application No. 62/515,960; Filed Jun. 6, 2017.

### FIELD OF THE INVENTION

This invention relates to a personal, disposable, ear/nose cleaning device which is capable of being used by a person to effectively clean his or her own ears/nose, or the ears/nose of others. The device is designed to have different sizes suitable for children and adults.

### BACKGROUND OF THE INVENTION

Buildup of mucus in the nostrils and wax in the ears can cause discomfort. It can be particularly challenging to clean wax/mucus buildup in babies, children, mentally or physically handicapped patients and elderly individuals who need care. When congested, abled children and adults have the ability to blow their nostrils to get some relief, and even so, sometimes that does not completely clean out mucus trapped in nasal passages. Also, adults capable of cleaning their ears sometimes have concerns of pushing earwax further into the ear canal.

The process of removing earwax or mucus especially in children could be risky because the wax or mucus could inadvertently be pushed further back into the canal and cause blockage. In fact, many professionals discourage the use of conventional cotton swabs or Q-tips to remove ear wax for the above reason. Also using the Q-tip to clean out nostrils especially in babies is difficult. There are multitude of devices used to clean ear wax or debris from ears and mucous from the nostrils of children, some disposable and some reusable. I tried a few of them when I had my kids and they were either too hard, pushed the wax further into the ear, dislodged in their ears or could not wrap mucus from their nostrils with ease. So, I developed a method to effectively clean the ears and nostrils of my children with much ease and less fuss. The invention will provide a simple to use device which can help parents improve the comfort of their child or caregivers improve the comfort of disabled or elderly patients.

After doing a patent search, the following patents had some similarities, yet major differences set my invention apart:
US patent NO. 2011/0054377 A1 relates to a nose cleaning device particularly for infants and young children. The device shaped like tweezers has buds on each end of its shaft formed from cotton or wool or other suitable soft material and their inner surfaces are substantially flat. The opposite end has a larger bud than the individual buds at the tip, and can be used as a regular cotton bud. Both ends comprise of tabs that extend outwardly as a depth limiting stop for insertion of the device.
U.S. Pat. No. 6,270,510 B1, describes a device for dislodging and removing mucus from nostrils, made of waxed paper or other twisted paper, with two distal padded ends. The ends have slightly raised loops to form scoops which may be hollow to form eyelets or closed to form pads or spoons.

### SUMMARY OF THE INVENTION

An ear/nose cleaning device for collecting mucus, wax and other materials/debris from the nostril and/ear canal. The device comprises of a piece of plastic framework with two arms/handles, pair of scoops and a U-shaped swab. The distal end of each arm has an oval-shaped scoop. The scoops are hollow inside with flat oval edges, which are tightly and lightly covered with cotton (or an absorbent material). The cotton or absorbent material is tightly and finely adhered to the scoops such that the insides of the scoops remain hollow and the outer portion smooth.

When these scoops are mechanically brought together by placing pressure on both arms they will grip and trap debris/mucus/wax in their hollow center. The two arms meet at the opposite/proximal end to form a swab in the shape of a “U”, which is slightly indented, also tightly and thinly wrapped/covered in cotton (or other absorbent material). The cotton (or other absorbent material) will be affixed to the ends using an adhesive that prevents unraveling of the cotton/material. The “U”—swab is designed for subsequent cleaning after the pair of scoops have been used to remove mucus, wax or other debris, and can also be used to remove smaller cerumen from the ear canal or absorb light mucus from the nostrils. The padding with thin cotton or other absorbent material is also to protect the ear canal and nasal canal while cleaning any debris or dirt. The lateral portion of each arm comprises of a grip portion or roughened surface, to make the device easy to grab and prevent slippage. The device is designed to be long enough not to get lodged in the nostrils or ear canal.

Different versions of the device can have different scoop sizes and different length, so that a device is used which is suitable for a particular size of child/adult. The device is designed as a single use disposable item, and is not designed to be cleaned during or after use.

### BRIEF DESCRIPTION OF THE DRAWINGS

FIG. 1 is a schematic illustration of the device in its open position at rest.

FIG. 2 is a schematic illustration of ear/nose cleaning device with a pair of scoops brought together.

FIG. 3A is a schematic illustration of the side view of the invention

FIG. 3B is a schematic illustration with a more detailed representation of a cross-sectional view of the scoop

FIG. 3C is a schematic illustration of the top view of one scoop

FIG. 4 is a schematic illustration of an alternate side view of ear/nose cleaning device.

FIG. 5 is a schematic illustration of the plastic framework of device, without cotton (or other absorbent material) attached to the scoops and U-shaped swab.

### DETAILED DESCRIPTION OF PREFERRED EMBODIMENTS

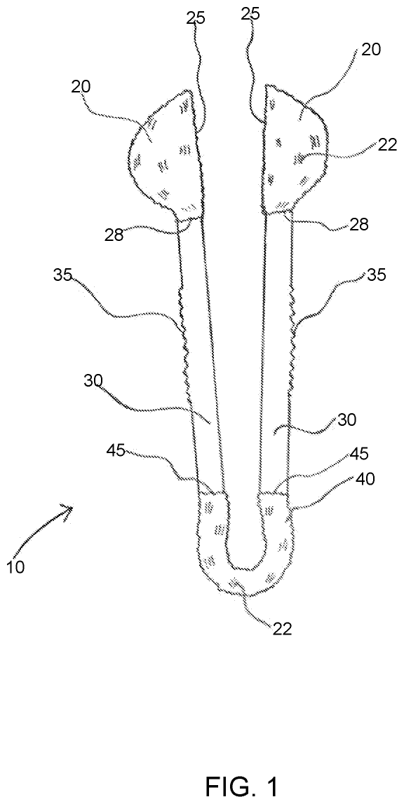

In FIG. 1, the ear/nose cleaning device 10 is at rest with two arms/handles 30 separated from each other. The device 10 is comprised of three main portions, a pair of scoops/bulbous-shaped portion 20, pair of arms/handles 30, and a U-shaped swab 40. The pair of plastic arms/handles 30 have scoops/bulbous portion 20 at the distal end also made of flexible plastic, and is tightly and lightly woven with cotton (or other absorbent material) 22, such that it leaves the edges 25 and outer surface flat/smooth. The pair of scoops or bulbous-shaped portions 20 have a hollow center 15 for grabbing and collecting ear wax/ear debris or nasal mucous. Both arms 30 meet proximally to form a U-shaped swab 40, also made of flexible plastic which is tightly and lightly woven with cotton (or other absorbent material) 22. Numeral 45 indicates the place where cotton (or other absorbent material) 22 on the U-shaped swab 40 is designed to stop. U-shaped swab 40 is designed to be slightly indented for easy removal of debris. The gripping of device 10 by a user is enhanced by the addition of a gripping/roughened portion 35 to the lateral portion of both arms 30.

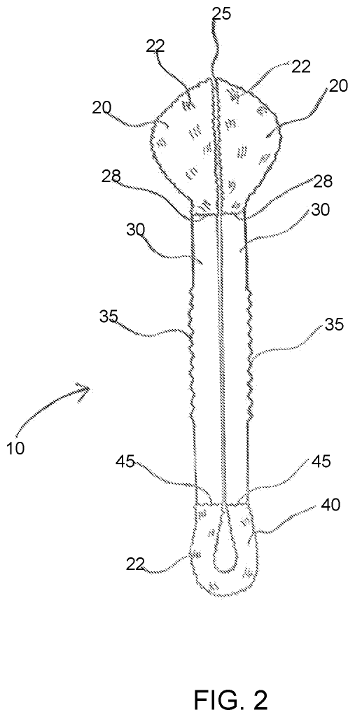

FIG. 2 shows device 10 closed, when pressure is applied to both arms 30. With the pair of scoops 20 brought together and their flat edges 25 in contact, a smooth continuous outer profile is formed, resembling a conventional single cotton bud, but with a hollow inside 15. The hollow inside 15 is designed to hold debris, wax or other material when scooped with pair of scoops 20.

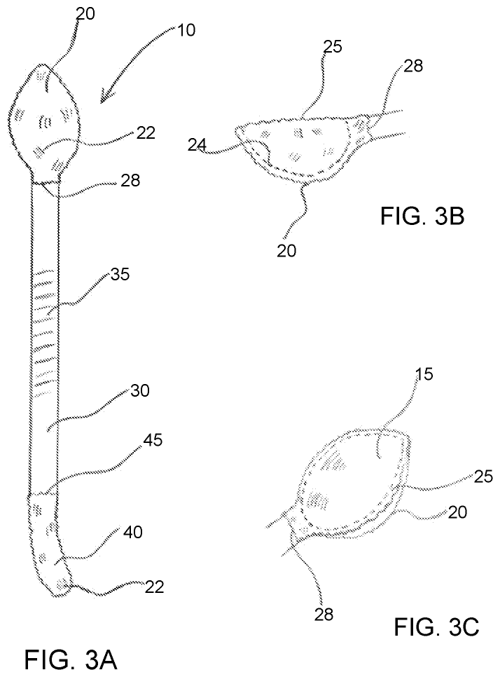

Device 10 is illustrated in FIG. 3A in side view such that only one arm 30 is visible, the other arm is hidden in view. The gripper/roughened portion 35 is schematically shown on the lateral portion of arm 30 in view. The gripping portion 35 is designed to help with gripping of device 10 when in use. Also shown is the lateral side of the U shaped swab 40, illustrating its slight curvature at the of the tip of the U shaped swab 40. The smooth other portion made of cotton (or other absorbent material) 22 can be seen, wherein the cotton (or other absorbent material) 22, tops 28 before the plastic arm 30 starts.

FIG. 3B is a cross-sectional side-view illustrating one scoop 20 with its hollow center 15 and flat border edges 25. Also schematically shown in FIG. 3B is the plastic framework 24 of the scoop 20 embedded within the cotton (or other absorbent material) 22.

FIG. 3C is a top view of scoops 20 further showing its hollow interior 15 and flat oval edges 25.

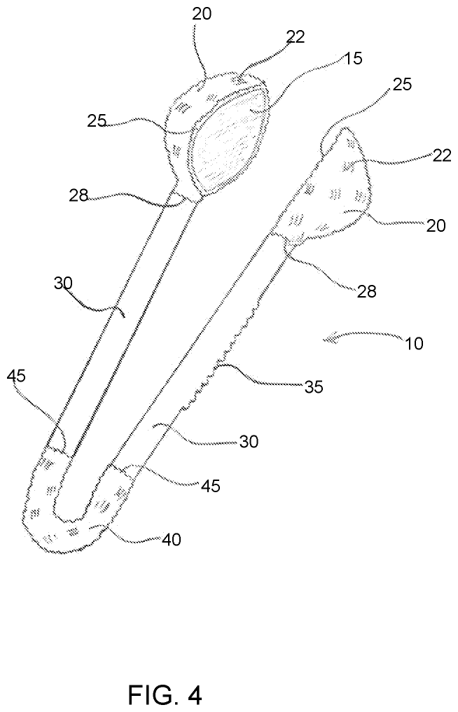

FIG. 4 illustrates an alternate side view of the scoop 20. The cotton (or other absorbent material) 22, is designed to make the scoops 20 feel soft and provide comfort to user. Without the cotton (or other absorbent material) 22 thinly and tightly adhered to the plastic framework 24 of scoops 20, the scoops 20 might feel rough/hard on user's skin in nostril or ear canal. As shown in FIG. 3, when a user applies pressure to arms 30, they will move inwards towards one another so that the pair of scoops 20 may be used to grip or grab objects there-between and keep them trapped in the hollow center 15. In this side view, only one of the gripping portion 35 is in view. Numeral 45 indicates the place where cotton (or other absorbent material) 22 on the U-shaped swab 40 is designed to stop.

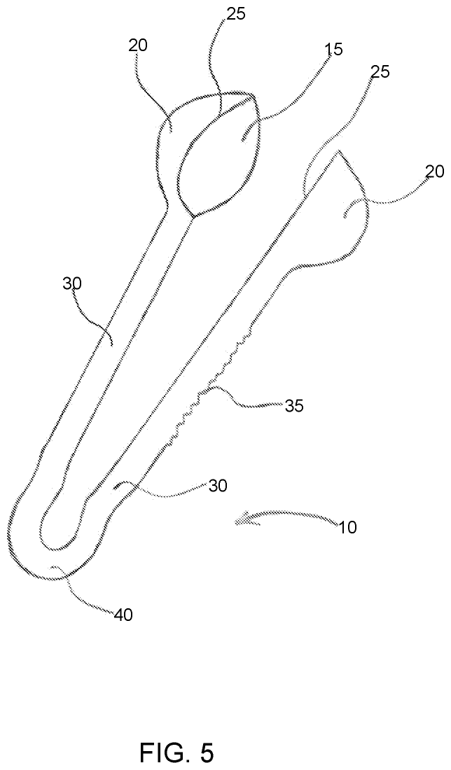

FIG. 5 is a schematic illustration of the plastic framework of device, without cotton (or other absorbent material) 22 attached to the scoops 20 and U-shaped swab 40. The plastic is to provide make device sturdy yet flexible. The device 10 in a lateral plane field such that only one grip/roughen portion 35 is visible.

1. An ear/nose cleaning device comprising:
One-piece plastic body framework;
a pair of arms/handles;
a gripping/roughen portion to the lateral sides of both arms;
a pair of scoops/bulbous-shaped portion at the distal end of the arms/handles;
the pair of scoops/bulbous-shaped portion have a hollow inside;
upper edges above the hollow insides for scraping and collecting ear wax/debris or nasal mucous and depositing within the hollow portion;
and U-shaped swab at proximal end of device;

2. An ear/nose cleaning in accordance with claim 1, wherein the pair of scoops/bulbous-shaped portions are comprising: a plastic body/framework; and cotton (or other absorbent material) tightly and finely adhered to plastic body/framework.

3. An ear/nose cleaning in accordance with claim 2, wherein scoops have a hollow inside and flat oval edges.

4. An ear/nose cleaning in accordance with claim 1, wherein the pair of arms/handles are comprising; gripping/roughened portion to the lateral sides of both arms

5. An ear/nose cleaning in accordance with claim 1, wherein the U-shaped swab is comprising: a plastic body/framework; cotton (or other absorbent material) tightly and finely adhered to plastic body/framework; and
a slight bend at the U-shaped swab for easy removal of debris.

6. An ear/nose cleaning device as claimed in any preceding claim, wherein with the scoops brought together in use may grip or grab debris/material there-between and keep them trapped in the hollow center.

7. An ear/nose cleaning in accordance with claim 1, wherein the device is disposable.
